(19) United States
(12) Patent Application Publication
(10) Pub. No.: US 2022/0113278 A1
(43) Pub. Date: Apr. 14, 2022

(54) GAS SENSOR

(71) Applicant: NGK INSULATORS, LTD., Nagoya (JP)

(72) Inventors: Yusuke WATANABE, Nagoya (JP); Shotaro NIIZUMA, Kasugai (JP); Hayami AOTA, Nagoya (JP); Toshihiro HIRAKAWA, Kasugai (JP)

(21) Appl. No.: 17/497,016

(22) Filed: Oct. 8, 2021

(30) Foreign Application Priority Data
Oct. 12, 2020 (JP) 2020-171674

(51) Int. Cl. G01N 27/407; G01N 33/00; G01N 27/409; G01N 27/41; G01N 27/30

(52) U.S. Cl. CPC G01N 27/4074; G01N 33/0037; G01N 27/301; G01N 27/41; G01N 27/409

(57) ABSTRACT

A gas sensor includes a pump electrode disposed in a measured gas flow path, an oxygen detection electrode disposed in the measured gas flow path and containing platinum and zirconia, and a reference electrode disposed in a reference gas chamber where a reference gas exists, and containing platinum and zirconia. A first position of a front end of the pump electrode is located closer to a rear end side than a second position of a front end of the oxygen detection electrode is. When the content of zirconia in the oxygen detection electrode is X [%], and a ratio of a distance between the first and second positions to a longitudinal dimension of the pump electrode is Y [%], Y≥141.96e^{-0.031X} is satisfied. The content of zirconia is not lower than that of platinum in the reference electrode.

### CROSS-REFERENCE TO RELATED APPLICATION

This application is based upon and claims the benefit of priority from Japanese Patent Application No. 2020-171674 filed on Oct. 12, 2020, the contents of which are incorporated herein by reference.

### BACKGROUND OF THE INVENTION

### Field of the Invention

The present invention relates to a gas sensor.

### Description of the Related Art

JP 2004-151018 A discloses a laminated gas sensor element capable of measuring the concentration of nitrogen oxide (NOₓ) or the like in a gas to be measured. The laminated gas sensor element disclosed in JP 2004-151018 A includes a first measured gas chamber, an oxygen pump cell, and a sensor cell. The measured gas is introduced into the first measured gas chamber. The oxygen pump cell has a pump electrode provided so as to face the first measured gas chamber. The sensor cell detects the concentration of a specific gas in the first measured gas chamber. The laminated gas sensor element disclosed in JP 2004-151018 A further includes a monitor cell. The monitor cell includes a monitor electrode facing the first measured gas chamber, and a monitor electrode facing the reference gas chamber.

### SUMMARY OF THE INVENTION

However, in the conventional gas sensor, the monitor electrode (reference electrode) facing the reference gas chamber may be peeled off. Further, in the conventional gas sensor, the monitor electrode (oxygen detection electrode) facing the first measured gas chamber may be peeled off. When the reference electrode, the oxygen detection electrode, and the like are peeled off, the detection accuracy is lowered, and further, detection may become impossible.

An object of the present invention is to provide a gas sensor capable of suppressing peeling of a reference electrode and an oxygen detection electrode.

According to an aspect of the present invention, provided is a gas sensor comprising: a measured gas flow path through which a measured gas introduced through a gas inlet flows, the gas inlet being located on a front end side which is one side; a pump electrode disposed in the measured gas flow path along a flow direction of the measured gas in the measured gas flow path; an oxygen detection electrode disposed in the measured gas flow path and containing platinum and zirconia; a reference electrode disposed in a reference gas chamber in which a reference gas exists, the reference electrode containing platinum and zirconia, wherein: when a position of a front end of the pump electrode is defined as a first position, and a position of a front end of the oxygen detection electrode is defined as a second position, the second position is located closer to a rear end side than the first position is, the rear end side being an opposite side to the front end side; when a content of zirconia in the oxygen detection electrode is defined as X [%], and a ratio of a distance between the first position and the second position to a longitudinal dimension of the pump electrode is defined as Y [%], Y≥141.96e^{-0.031X} is satisfied; and a content of zirconia in the reference electrode is equal to or higher than a content of platinum in the reference electrode.

According to the present invention, it is possible to provide a gas sensor capable of suppressing peeling of a reference electrode and an oxygen detection electrode.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a cross-sectional view showing an example of a gas sensor according to an embodiment;

FIG. 2 is a cross-sectional view showing a part of the gas sensor according to the embodiment;

FIG. 3 is a graph showing the distribution of oxygen concentration;

FIG. 4 is a cross-sectional view showing another example of the gas sensor according to the embodiment;

FIG. 5 is a cross-sectional view showing still another example of the gas sensor according to the embodiment;

FIG. 6 is a cross-sectional view showing yet another example of the gas sensor according to the embodiment;

FIG. 7 is a plan view corresponding to a part of FIG. 6;

FIG. 8 is a diagram showing Table 1 illustrating test results; and

FIG. 9 is a graph showing test results.

### DESCRIPTION OF THE PREFERRED EMBODIMENTS

The gas sensor according to the present invention will be described below in detail in connection with a preferred embodiment while referring to the accompanying drawings.

### [Embodiment]

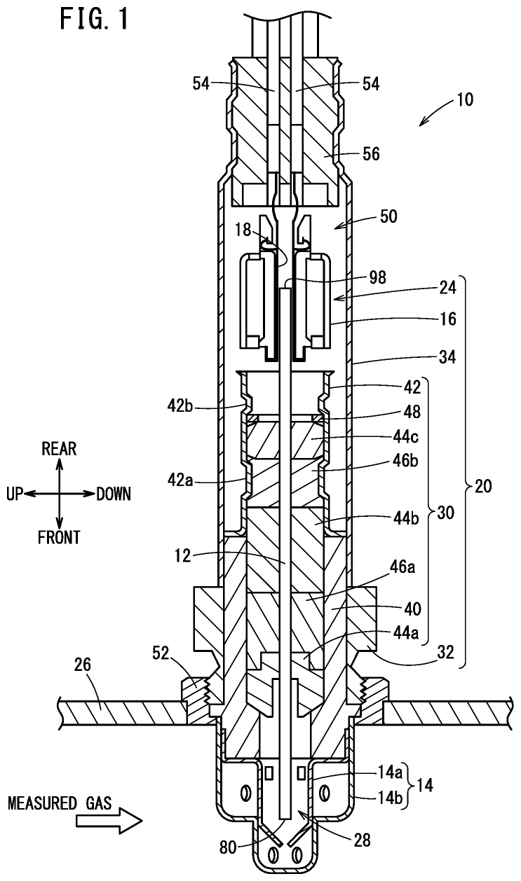

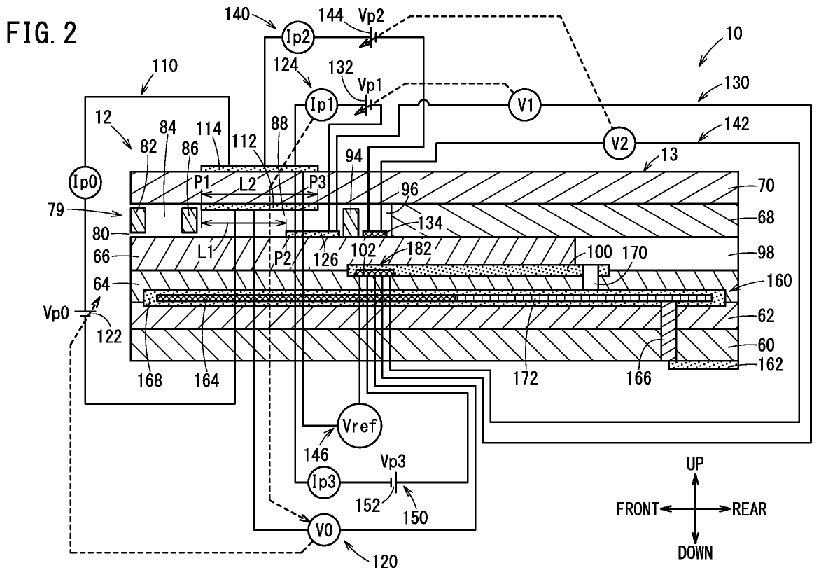

A gas sensor according to an embodiment will be described with reference to FIGS. 1 to 9. FIG. 1 is a cross-sectional view showing an example of a gas sensor according to the present embodiment. FIG. 2 is a cross-sectional view showing a part of the gas sensor according to the present embodiment.

As shown in FIG. 1, a gas sensor 10 includes a sensor element 12. The sensor element 12 has, for example, an elongated rectangular parallelepiped shape. The longitudinal direction of the sensor element 12 is defined as a front-rear direction. That is, the left-right direction in FIG. 2 is defined as the front-rear direction. The thickness direction of the sensor element 12 is defined as an up-down direction. That is, the up-down direction in FIG. 2 is defined as the up-down direction. The width direction of the sensor element 12 is defined as the left-right direction. That is, a direction perpendicular to the front-rear direction and the up-down direction is defined as the left-right direction.

The gas sensor 10 further includes a protective cover 14. The protective cover 14 protects the front end side which is one side in the longitudinal direction of the sensor element 12. The gas sensor 10 further includes a sensor assembly 20 including a ceramic housing 16. Metal terminals 18 are attached to the ceramic housing 16. The metal terminals 18 hold the rear end portion of the sensor element 12 and are electrically connected to the sensor element 12. The metal terminals 18 are attached to the ceramic housing 16 to form a connector 24.

The gas sensor 10 may be attached to a pipe 26, for example. Examples of the pipe 26 include an exhaust gas pipe of a vehicle. The gas sensor 10 can be used to measure the concentration of a specific gas contained in an exhaust gas or the like, which is a measured gas. Examples of the specific gas include, but are not limited to, nitrogen oxides, oxygen (O₂), and the like.

The protective cover 14 includes an inner protective cover 14a and an outer protective cover 14b. The inner protective cover 14a is a bottomed tubular protective cover that covers the front end of the sensor element 12. The outer protective cover 14b is a bottomed tubular protective cover that covers the inner protective cover 14a. The inner protective cover 14a and the outer protective cover 14b have formed therein a plurality of holes that allow the measured gas to flow in the interior of the protective cover 14. The front end of the sensor element 12 is located in a space surrounded by the inner protective cover 14a. That is, the front end of the sensor element 12 is located in a sensor element chamber 28.

The sensor assembly 20 includes an element sealing body 30 for sealing and fixing the sensor element 12. The sensor assembly 20 further includes a nut 32 attached to the element sealing body 30. The sensor assembly 20 further includes an outer tube 34 and the connector 24. The metal terminals 18 provided in the connector 24 are connected to electrodes (not shown) formed on the surfaces of the rear end of the sensor element 12. That is, the metal terminals 18 provided in the connector 24 are connected to the electrodes (not shown) formed on the upper surface and the lower surface of the rear end of the sensor element 12.

The element sealing body 30 includes a tubular main fitting 40 and a tubular inner tube 42. The central axis of the main fitting 40 and the central axis of the inner tube 42 coincide with each other. The main fitting 40 and the inner tube 42 are fixed by welding. Ceramic supporters 44a to 44c, green compacts 46a and 46b, and a metal ring 48 are sealed in a through hole inside the main fitting 40 and the inner tube 42. The sensor element 12 is located on the central axis of the element sealing body 30. The sensor element 12 penetrates the element sealing body 30 in the front-rear direction. The inner tube 42 has reduced-diameter portions 42a and 42b. The reduced-diameter portion 42a presses the green compact 46b in a direction toward the central axial of the inner tube 42. The reduced-diameter portion 42b presses forward the ceramic supporters 44a to 44c and the green compacts 46a and 46b via the metal ring 48. The green compacts 46a and 46b are compressed between the main fitting 40 and the sensor element 12 and between the inner tube 42 and the sensor element 12 by the pressing forces from the reduced-diameter portions 42a and 42b. Thus, the green compacts 46a and 46b provide a seal between the sensor element chamber 28 in the protective cover 14 and a space 50 in the outer tube 34, and fix the sensor element 12.

The nut 32 is fixed to the main fitting 40. The central axis of the nut 32 and the central axis of the main fitting 40 coincide with each other. A male screw portion is formed on an outer peripheral surface of the nut 32. A female screw portion is formed on an inner peripheral surface of a fixing member 52 welded to the pipe 26. The male screw portion formed on the outer peripheral surface of the nut 32 is inserted into the fixing member 52 having the female screw portion formed on the inner peripheral surface thereof. Thus, the gas sensor 10 is fixed to the pipe 26 in a state where the front end of the sensor element 12 protected by the protective cover 14 protrudes into the pipe 26.

The outer tube 34 encloses the inner tube 42, the sensor element 12, and the connector 24. A plurality of lead wires 54 connected to the connector 24 are drawn out from the rear end of the outer tube 34 to the outside. The lead wires 54 electrically conduct to electrodes of the sensor element 12 via the connector 24. The gap between the outer tube 34 and the lead wires 54 is sealed by an elastic insulating member 56 formed of a grommet or the like. The space 50 in the outer tube 34 is filled with a reference gas (atmosphere). The rear end of the sensor element 12 is located in the space 50.

As shown in FIG. 2, the sensor element 12 includes a laminate 13 formed of a first substrate layer 60, a second substrate layer 62, a third substrate layer 64, a solid electrolyte layer 66, a spacer layer 68, and a solid electrolyte layer 70. The second substrate layer 62 is laminated on the first substrate layer 60. The third substrate layer 64 is laminated on the second substrate layer 62. The solid electrolyte layer 66 is laminated on the third substrate layer 64. The spacer layer 68 is laminated on the solid electrolyte layer 66. The solid electrolyte layer 70 is laminated on the spacer layer 68. For example, a solid electrolyte is used as the material of these layers 60, 62, 64, 66, 68, and 70. More specifically, an oxygen ion conductive solid electrolyte is used as the material of these layers 60, 62, 64, 66, 68, and 70. Examples of the oxygen ion conductive solid electrolyte include zirconia (ZrO₂). These layers 60, 62, 64, 66, 68, 70 are highly airtight. The sensor element 12 can be manufactured as follows. Specifically, predetermined processing, printing of predetermined patterns, and the like are performed on ceramic green sheets corresponding to the respective layers. Thereafter, these ceramic green sheets are laminated. Then, these ceramic green sheets are integrated by firing. In this way, the sensor element 12 can be manufactured. The material of these layers 60, 62, 64, 66, 68, and 70 is not limited to the solid electrolyte. For example, the spacer layer 68 may be an insulator layer or the like. Examples of the insulator layer include alumina and the like.

A measured gas flow path (measured gas flow portion) 79 through which the measured gas flows is formed inside the sensor element 12. The flow direction of the measured gas in the measured gas flow path 79 is the longitudinal direction of the measured gas flow path 79. The measured gas flow path 79 is formed in the spacer layer 68. That is, the measured gas flow path 79 is formed by hollowing out a part of the spacer layer 68. The side surface of the measured gas flow path 79 is defined by the spacer layer 68. The bottom surface (lower surface) of the measured gas flow path 79 is defined by the upper surface of the solid electrolyte layer 66. The top surface (upper surface) of the measured gas flow path 79 is defined by the lower surface of the solid electrolyte layer 70. One end of the measured gas flow path 79 is a gas inlet 80 through which the measured gas is introduced. That is, the gas inlet 80 is on the left side of FIG. 2. The gas inlet 80 is located on the front end side which is one side in the longitudinal direction of the sensor element 12. That is, the gas inlet 80 is located on the front end side which is one side in the longitudinal direction of the laminate 13.

In the measured gas flow path 79, a diffusion control portion 82 is provided at the rear stage of the gas inlet 80. The diffusion control portion 82 includes, for example, two slits. The longitudinal direction of the slits is, for example, a direction perpendicular to the drawing sheet of FIG. 2. A buffer space (internal cavity) 84 is provided at the rear stage of the diffusion control portion 82. A diffusion control portion 86 is provided at the rear stage of the buffer space 84. In this way, the buffer space 84 is defined by the diffusion control portion 82 and the diffusion control portion 86. The diffusion control portion 86 includes, for example, two slits. The longitudinal direction of the slits is, for example, a direction perpendicular to the drawing sheet of FIG. 2. An internal cavity 88 is provided at the rear stage of the diffusion control portion 86. The internal cavity 88 communicates with the buffer space 84 via the diffusion control portion 86. A diffusion control portion 94 is provided at the rear stage of the internal cavity 88. The internal cavity 88 is thus defined by the diffusion control portion 86 and the diffusion control portion 94. The diffusion control portion 94 includes, for example, one slit. The longitudinal direction of the slit is, for example, a direction perpendicular to the drawing sheet of FIG. 2. An internal cavity 96 is provided at the rear stage of the diffusion control portion 94. The internal cavity 96 communicates with the internal cavity 88 via the diffusion control portion 94. Thus, the internal cavity 96 is defined by the diffusion control portion 94. At least one of the diffusion control portions 82, 86, and 94 may be formed of a porous body.

A reference gas introduction space 98 is formed inside the sensor element 12. The measured gas flow path 79 described above is located on one side in the longitudinal direction of the sensor element 12. That is, the measured gas flow path 79 is located on the front end side of the sensor element 12. The reference gas introduction space 98 is located on the other side in the longitudinal direction of the sensor element 12. That is, the reference gas introduction space 98 is located on the rear end side of the sensor element 12. The reference gas introduction space 98 is formed by hollowing out a part of the solid electrolyte layer 66. The side surface of the reference gas introduction space 98 is defined by the solid electrolyte layer 66. The lower surface of the reference gas introduction space 98 is defined by the upper surface of the third substrate layer 64. The upper surface of the reference gas introduction space 98 is defined by the lower surface of the spacer layer 68. A reference gas can be introduced into the reference gas introduction space 98. The atmosphere in the space 50 (see FIG. 1) can be the reference gas. The reference gas for measuring the concentration of nitrogen oxide is, for example, atmospheric air.

An atmosphere introduction layer 100 is provided inside the sensor element 12. The atmosphere introduction layer 100 is provided, for example, between the third substrate layer 64 and the solid electrolyte layer 66. A porous material is used as the material of the atmosphere introduction layer 100. More specifically, for example, porous ceramics such as porous alumina can be used as the material of the atmosphere introduction layer 100. A part of the atmosphere introduction layer 100 is exposed in the reference gas introduction space 98. A reference gas can be introduced into the atmosphere introduction layer 100 through the reference gas introduction space 98. The atmosphere introduction layer 100 is formed so as to cover a reference electrode 102 described later. The atmosphere introduction layer 100 allows the reference gas in the reference gas introduction space 98 to reach the reference electrode 102 while applying a predetermined diffusion resistance to the reference gas. A rear end portion of the atmosphere introduction layer 100 is exposed in the reference gas introduction space 98. A portion which covers the reference electrode 102, of the atmosphere introduction layer 100 is not exposed in the reference gas introduction space 98.

The reference electrode 102 is formed on the upper surface of the third substrate layer 64. The reference electrode 102 is formed directly on the third substrate layer 64. A part of the reference electrode 102 is exposed in a reference gas chamber 182 in which the reference gas exists. An atmosphere introduction layer 100 exists in the reference gas chamber 182. The portion of the reference electrode 102 other than the portion in contact with the third substrate layer 64 is covered with the atmosphere introduction layer 100. Here, the case where the atmosphere introduction layer 100 exists in the reference gas chamber 182 will be described as an example, but the atmosphere introduction layer 100 may not exist in the reference gas chamber 182. That is, the reference gas chamber 182 may be empty. The atmosphere introduction layer 100 is formed so as to reach the reference gas introduction space 98. The reference gas chamber 182 may contain a reference gas introduced through the atmosphere introduction layer 100. As will be described later, the oxygen concentration (oxygen partial pressure) in the internal cavity 88 and the oxygen concentration in the internal cavity 96 can be measured using the reference electrode 102. For example, porous cermet can be used as the material of the reference electrode 102. Cermet is a composite material of ceramic and metal. For example, cermet of platinum (Pt) and zirconia may be used as the material of the reference electrode 102.

In this embodiment, the content of zirconia in the reference electrode 102 is set to be relatively high. More specifically, in the present embodiment, the content of zirconia in the reference electrode 102 is set to be equal to or higher than the content of platinum in the reference electrode 102.

In the present embodiment, the content of zirconia in the reference electrode 102 is set to be equal to or higher than the content of platinum in the reference electrode 102 for the following reason. That is, in order to maintain the measurement accuracy of the gas sensor 10, oxygen may be pumped in by applying a voltage between an outer pump electrode 114 or the like and the reference electrode 102. When oxygen is pumped in, the oxygen concentration temporarily increases around the reference electrode 102 and in the reference gas chamber 182. While the gas sensor 10 is repeatedly used over a long period of time, platinum contained in the reference electrode 102 is oxidized to form platinum oxide. In a severe use environment such as a high temperature, platinum is more likely to be oxidized, and thus platinum oxide is more likely to be generated. Platinum oxide is more likely to sublime than platinum. Therefore, when platinum oxide is generated in the reference electrode 102, the platinum oxide may sublime and peeling may occur at the interface between the reference electrode 102 and the third substrate layer 64. On the other hand, zirconia does not sublime unless at a significantly high temperature. Therefore, if the content of zirconia in the reference electrode 102 is set to be relatively high, the amount of sublimation of the material of the reference electrode 102 becomes small, and as a result, peeling of the reference electrode 102 can be suppressed. That is, if the content of platinum in the reference electrode 102 is set to be relatively low, the amount of sublimation of the material of the reference electrode 102 becomes small, and as a result, peeling of the reference electrode 102 can be suppressed. For this reason, in the present embodiment, the content of zirconia in the reference electrode 102 is set to be equal to or higher than the content of platinum in the reference electrode 102.

The gas inlet 80 is open to the external space. The measured gas can be taken into the sensor element 12 from the external space through the gas inlet 80. The diffusion control portion 82 applies a predetermined diffusion resistance to the measured gas taken in from the gas inlet 80. The buffer space 84 guides the measured gas introduced by the diffusion control portion 82, to the diffusion control portion 86. The diffusion control portion 86 applies a predetermined diffusion resistance to the measured gas introduced from the buffer space 84 into the internal cavity 88. The measured gas taken into the sensor element 12 through the gas inlet 80 is introduced into the internal cavity 88 through the diffusion control portion 82, the buffer space 84, and the diffusion control portion 86. There is a case where the measured gas is rapidly taken into the sensor element 12 due to pressure fluctuation in the external space. In the case where the measured gas is an automobile exhaust gas, the pressure fluctuation corresponds to the exhaust pressure pulsation. Even when the measured gas is rapidly taken into the sensor element 12 due to the pressure fluctuation in the external space, the concentration fluctuation of the measured gas is canceled while the measured gas passes through the diffusion control portion 82, the buffer space 84, and the diffusion control portion 86. Since the measured gas in which the concentration fluctuation is canceled is introduced into the internal cavity 88, the concentration fluctuation of the measured gas introduced into the internal cavity 88 is almost negligible. The internal cavity 88 is a space for adjusting the oxygen partial pressure in the measured gas introduced thereto via the diffusion control portion 86. The oxygen partial pressure can be adjusted by operation of a main pump cell 110 described later.

The sensor element 12 further includes the main pump cell 110. The main pump cell 110 is an electrochemical pump cell formed of a pump electrode 112, the outer pump electrode 114, and the solid electrolyte layer 70 sandwiched between the pump electrode 112 and the outer pump electrode 114. The pump electrode 112 is disposed in the measured gas flow path 79 so as to extend along the flow direction of the measured gas in the measured gas flow path 79. The outer pump electrode 114 is disposed outside the laminate 13. The pump electrode 112 is formed on the inner surface of the internal cavity 88. The outer pump electrode 114 is formed on the upper surface of the solid electrolyte layer 70. The outer pump electrode 114 is formed in a region corresponding to a region where the pump electrode 112 is formed. The outer pump electrode 114 is exposed to the external space. That is, the outer pump electrode 114 is exposed in the sensor element chamber 28 in FIG. 1.

The planar shape of the pump electrode 112 is, for example, rectangular. The pump electrode 112 is formed on one of the bottom surface of the internal cavity 88 and the top surface of the internal cavity 88. Note that an oxygen detection electrode 126 described later is formed on the other of the bottom surface of the internal cavity 88 and the top surface of the internal cavity 88. FIG. 2 shows an example in which the pump electrode 112 is formed on the top surface of the internal cavity 88. That is, FIG. 2 shows an example in which the pump electrode 112 is formed on the lower surface of the solid electrolyte layer 70. The longitudinal direction of the pump electrode 112 coincides with the longitudinal direction of the internal cavity 88.

As the material of the pump electrode 112 and the outer pump electrode 114, for example, a porous cermet can be used. For example, a cermet of platinum and zirconia containing 1% of gold (Au) can be used as the material of the pump electrode 112 and the outer pump electrode 114. As the material of the pump electrode 112 in contact with the measured gas, it is preferable to use a material whose reducing power for nitrogen oxide in the measured gas is weakened. The cermet of platinum and zirconia containing 1% of gold is a material whose reducing power for nitrogen oxide in the measured gas is weakened.

In the main pump cell 110, when a desired pump voltage Vp0 is applied across the pump electrode 112 and the outer pump electrode 114, a pump current Ip0 flows between the pump electrode 112 and the outer pump electrode 114 in the positive direction or negative direction. Accordingly, oxygen in the internal cavity 88 can be pumped out to the external space, or oxygen in the external space can be pumped into the internal cavity 88.

The sensor element 12 further includes an oxygen-partial-pressure detection sensor cell (main-pump-controlling oxygen-partial-pressure detection sensor cell) 120. The oxygen-partial-pressure detection sensor cell 120 is an electrochemical sensor cell for detecting the oxygen concentration (oxygen partial pressure) in the atmosphere in the internal cavity 88. The oxygen-partial-pressure detection sensor cell 120 is formed of the pump electrode 112, the solid electrolyte layers 66 and 70, the spacer layer 68, and the reference electrode 102.

By detecting an electromotive force V0 in the oxygen-partial-pressure detection sensor cell 120, the oxygen concentration in the atmosphere in the internal cavity 88 can be ascertained. Further, the pump current Ip0 can be controlled by feedback controlling the pump voltage Vp0 of a variable power supply 122 so that the electromotive force V0 is kept constant. Thus, the oxygen concentration in the internal cavity 88 can be maintained at a predetermined constant value. In this way, the oxygen concentration can be adjusted.

The sensor element 12 further includes an auxiliary pump cell 124. The auxiliary pump cell 124 is an auxiliary electrochemical pump cell. The auxiliary pump cell 124 can further adjust the oxygen concentration of the measured gas whose oxygen concentration has been adjusted in advance by the main pump cell 110. Since the oxygen concentration is kept constant with high accuracy by the auxiliary pump cell 124, the gas sensor 10 can measure the concentration of nitrogen oxide with high accuracy. The auxiliary pump cell 124 is formed of the oxygen detection electrode 126 that can function also as an auxiliary pump electrode, the outer pump electrode 114, and the solid electrolyte layer 70. The oxygen detection electrode 126 is formed on the inner surface of the internal cavity 88. Note that an outer electrode provided separately from the outer pump electrode 114 may be used for the auxiliary pump cell 124.

The pump electrode 112 and the oxygen detection electrode 126 are disposed in the same internal cavity 88. As described above, the pump electrode 112 is formed on one of the bottom surface of the internal cavity 88 and the top surface of the internal cavity 88. The oxygen detection electrode 126 is formed on the other of the bottom surface of the internal cavity 88 and the top surface of the internal cavity 88. FIG. 2 shows an example in which the oxygen detection electrode 126 is formed on the bottom surface of the internal cavity 88. In other words, FIG. 2 shows an example in which the oxygen detection electrode 126 is formed on the upper surface of the solid electrolyte layer 66. The longitudinal direction of the oxygen detection electrode 126 coincides with the longitudinal direction of the internal cavity 88. Like the pump electrode 112, the oxygen detection electrode 126 is preferably made of a material whose reducing power for nitrogen oxide in the measured gas is weakened.

In the auxiliary pump cell 124, when a voltage Vp1 is applied across the oxygen detection electrode 126, which can function also as an auxiliary pump electrode, and the outer pump electrode 114 by a variable power supply 132, the following occurs. That is, a pump current Ip1 flows between the oxygen detection electrode 126 and the outer pump electrode 114 in the positive direction or negative direction. Accordingly, oxygen in the internal cavity 88 can be pumped out to the external space, or oxygen in the external space can be pumped into the internal cavity 88.

The sensor element 12 further includes an oxygen-partial-pressure detection sensor cell (auxiliary-pump-controlling oxygen-partial-pressure detection sensor cell) 130. The oxygen-partial-pressure detection sensor cell 130 is an electrochemical sensor cell for controlling the oxygen concentration in the atmosphere in the internal cavity 88. The oxygen-partial-pressure detection sensor cell 130 is formed of the oxygen detection electrode 126, the reference electrode 102, the solid electrolyte layers 66 and 70, and the spacer layer 68.

The voltage Vp1 is controlled based on an electromotive force V1 detected by the oxygen-partial-pressure detection sensor cell 130. As described above, in the auxiliary pump cell 124, the pump current Ip1 flows between the oxygen detection electrode 126 and the outer pump electrode 114 in accordance with the voltage Vp1 applied across the oxygen detection electrode 126, which can function also as an auxiliary pump electrode, and the outer pump electrode 114. Thus, pumping of oxygen can be performed. In this manner, the oxygen partial pressure in the atmosphere in the internal cavity 88 can be controlled to such low partial pressure as not to substantially affect the measurement of the concentration of nitrogen oxide.

A signal indicating the pump current Ip1 can be input to the oxygen-partial-pressure detection sensor cell 120. The oxygen-partial-pressure detection sensor cell 120 controls a signal indicating the electromotive force V0 based on the signal indicating the pump current Ip1. In the case where the gas sensor 10 is used as a gas sensor that measures the concentration of nitrogen oxide, the oxygen concentration in the atmosphere in the internal cavity 88 can be set to a constant value of, for example, about 0.001 ppm by the action of the main pump cell 110 and the auxiliary pump cell 124.

A second position P2, which is the position of the end portion of the oxygen detection electrode 126 on the front end side, is located closer to the rear end side than a first position P1, which is the position of the end portion of the pump electrode 112 on the front end side, is. The reason why the second position P2 is located closer to the rear end side than the first position P1 is, is to further adjust, by the auxiliary pump cell 124, the oxygen concentration of the measured gas whose oxygen concentration has been adjusted in advance by the main pump cell 110.

When the oxygen detection electrode 126 is repeatedly used over a long period of time, platinum contained in the oxygen detection electrode 126 may be oxidized to form platinum oxide. As described above, in a severe use environment such as a high temperature, platinum is more likely to be oxidized, and thus platinum oxide is more likely to be generated. Platinum oxide is more likely to sublime than platinum. Therefore, when platinum oxide is generated in the oxygen detection electrode 126, the platinum oxide may sublime and peeling may occur at the interface between the oxygen detection electrode 126 and the solid electrolyte layer 66.

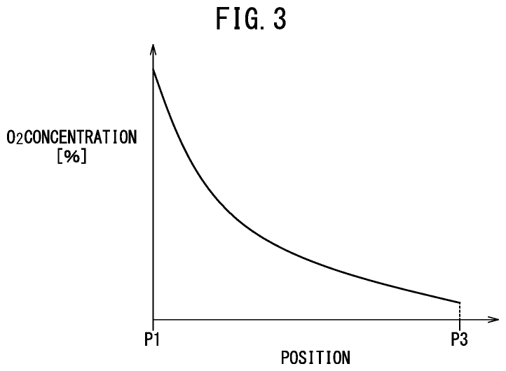

FIG. 3 is a graph showing the distribution of oxygen concentration. The horizontal axis in FIG. 3 indicates the position in the measured gas flow path 79. P1 in FIG. 3 corresponds to the first position P1 (see FIG. 2) which is the position of the end portion of the pump electrode 112 on the front end side. P3 in FIG. 3 corresponds to a third position P3 (see FIG. 2) which is the position of the end portion of the pump electrode 112 on the rear end side. As can be seen from FIG. 3, the oxygen concentration gradually decreases from the first position P1 toward the third position P3. The oxygen concentration gradually decreases because oxygen is pumped out to the external space by the main pump cell 110.

When the content of platinum in the oxygen detection electrode 126 is relatively high, the amount of platinum oxide generated by the oxidation of platinum can also be relatively large. In the case where a relatively large amount of platinum oxide is generated, the amount of loss of the constituent elements of the oxygen detection electrode 126 when the platinum oxide sublimes also increases, and peeling of the oxygen detection electrode 126 becomes more likely to occur. Therefore, when the content of platinum in the oxygen detection electrode 126 is relatively high, positioning the oxygen detection electrode 126 at a site where the oxygen concentration becomes sufficiently low by the operation of the main pump cell 110 contributes to suppression of peeling of the oxygen detection electrode 126. That is, when the content of platinum in the oxygen detection electrode 126 is relatively high, it is preferable to sufficiently increase a distance L1 between the first position P1 and the second position P2. As described above, the first position P1 is the position of the end portion of the pump electrode 112 on the front end side. As described above, the second position P2 is the position of the end portion of the oxygen detection electrode 126 on the front end side.

On the other hand, when the content of platinum in the oxygen detection electrode 126 is relatively low, the amount of platinum oxide generated by the oxidation of platinum is also relatively small. In the case where a relatively small amount of platinum oxide is generated, the amount of loss of the constituent elements of the oxygen detection electrode 126 when the platinum oxide sublimes is also small, and therefore, peeling of the oxygen detection electrode 126 is less likely to occur. For this reason, in the case where the content of platinum in the oxygen detection electrode 126 is relatively low, even if the oxygen detection electrode 126 is positioned at a site where the oxygen concentration is relatively high, peeling of the oxygen detection electrode 126 is less likely to occur. That is, when the content of platinum in the oxygen detection electrode 126 is relatively low, the distance L1 between the first position P1, which is the position of the end portion of the pump electrode 112 on the front end side, and the second position P2, which is the position of the end portion of the oxygen detection electrode 126 on the front end side, may be relatively small.

As a result of performing a peeling test as described later, the inventors of the present application have found that it is preferable that the positional relationship between the pump electrode 112 and the oxygen detection electrode 126, and the content (volume content) of zirconia in the oxygen detection electrode 126 are set so as to satisfy the condition represented by the following expression (1).

Y≥141.96e^{-0.031X} (1)

X [%] is the content of zirconia in the oxygen detection electrode 126. Y [%] is a ratio (L1/L2) of the distance L1 between the first position P1 and the second position P2 to a longitudinal dimension L2 of the pump electrode 112.

In addition, as a result of performing a peeling test as described later, the inventors of the present application have found that it is more preferable to satisfy the condition represented by the following expression (2).

Y≥2645.5X^{-1.024} (2)

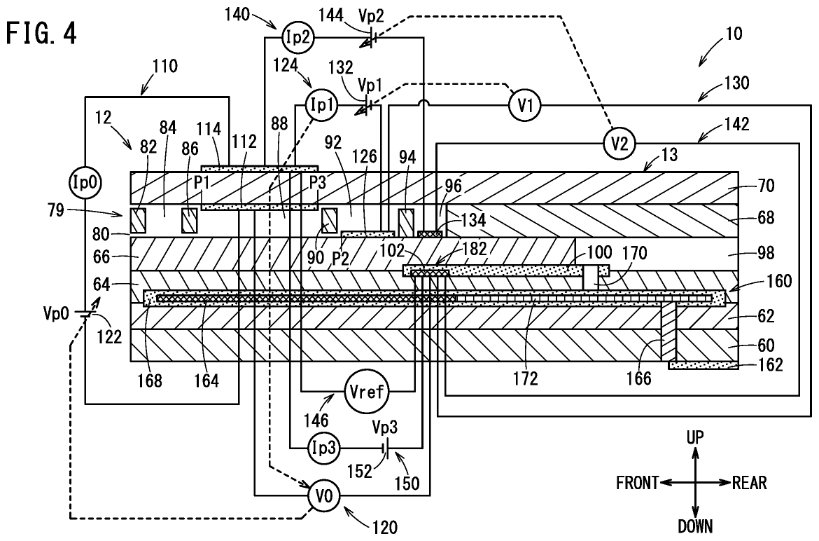

In the configuration shown in FIG. 4 described later, Y is greater than 100%, but in the configuration shown in FIG. 2, Y can be set to an arbitrary value.

The content of zirconia in the oxygen detection electrode 126 is preferably 90% or less. This is because, when the content of zirconia in the oxygen detection electrode 126 is excessively high, the content of platinum in the oxygen detection electrode 126 becomes excessively low, and the oxygen concentration or the like cannot be detected satisfactorily.

The content of platinum in the oxygen detection electrode 126 is preferably higher than the content of zirconia in the oxygen detection electrode 126. This is because the relatively high content of platinum in the oxygen detection electrode 126 can contribute to an improvement in the response speed of the gas sensor 10.

The diffusion control portion 94 applies a predetermined diffusion resistance to the measured gas introduced from the internal cavity 88 to the internal cavity 96, and guides the measured gas to the internal cavity 96. As described above, the oxygen concentration in the atmosphere in the internal cavity 88 can be controlled by the main pump cell 110 and the auxiliary pump cell 124. The diffusion control portion 94 applies a predetermined diffusion resistance to the measured gas whose oxygen concentration has been controlled by the main pump cell 110 and the auxiliary pump cell 124. The diffusion control portion 94 also serves to limit the amount of nitrogen oxides flowing into the internal cavity 96.

The measured gas whose oxygen concentration has been adjusted in advance in the internal cavity 88 is introduced into the internal cavity 96 via the diffusion control portion 94. The internal cavity 96 is a space for detecting the concentration of nitrogen oxide in the measured gas. That is, the internal cavity 96 is a space for detecting the concentration of nitrogen oxide. The concentration of nitrogen oxide can be measured by operating a measurement pump cell 140 described later.

The sensor element 12 further includes the measurement pump cell 140. The measurement pump cell 140 is an electrochemical pump cell for measuring the concentration of nitrogen oxide in the measured gas introduced into the internal cavity 96. The measurement pump cell 140 is formed of a nitrogen oxide detection electrode 134, the outer pump electrode 114, the solid electrolyte layers 66 and 70, and the spacer layer 68. The nitrogen oxide detection electrode (measurement electrode) 134 is formed on the upper surface of the solid electrolyte layer 66. As the material of the nitrogen oxide detection electrode 134, for example, porous cermet can be used. The nitrogen oxide detection electrode 134 functions as a catalyst for reducing nitrogen oxide present in the atmosphere in the internal cavity 96.

The measurement pump cell 140 pumps out oxygen generated by decomposition of nitrogen oxide in the atmosphere around the nitrogen oxide detection electrode 134. A pump current Ip2 corresponding to the amount of oxygen pumped out by the measurement pump cell 140 can be detected.

The sensor element 12 further includes an oxygen-partial-pressure detection sensor cell (measurement-pump-controlling oxygen-partial-pressure detection sensor cell) 142. The oxygen-partial-pressure detection sensor cell 142 is an electrochemical sensor cell for detecting the oxygen partial pressure around the nitrogen oxide detection electrode 134. The oxygen-partial-pressure detection sensor cell 142 is formed of the solid electrolyte layer 66, the nitrogen oxide detection electrode 134, and the reference electrode 102. A variable power supply 144 can be controlled based on an electromotive force V2 detected by the oxygen-partial-pressure detection sensor cell 142.

The measured gas whose oxygen partial pressure has been controlled in the internal cavity 88 reaches the nitrogen oxide detection electrode 134 in the internal cavity 96 via the diffusion control portion 94. The nitrogen oxide in the measured gas around the nitrogen oxide detection electrode 134 is reduced by the nitrogen oxide detection electrode 134 (2NO→N₂+O2), and oxygen is generated around the nitrogen oxide detection electrode 134. The generated oxygen is pumped by the measurement pump cell 140. At this time, the voltage Vp2 of the variable power supply 144 is controlled so that the electromotive force V2 detected by the oxygen-partial-pressure detection sensor cell 142 is kept constant. The amount of oxygen generated around the nitrogen oxide detection electrode 134 is proportional to the concentration of nitrogen oxide in the measured gas. Therefore, the concentration of the nitrogen oxide in the measured gas can be calculated based on the pump current Ip2 in the measurement pump cell 140.

The sensor element 12 further includes a sensor cell 146. The sensor cell 146 is an electrochemical sensor cell formed of the third substrate layer 64, the solid electrolyte layers 66 and 70, the spacer layer 68, the outer pump electrode 114, and the reference electrode 102. The oxygen partial pressure in the measured gas outside the sensor element 12 can be detected based on an electromotive force Vref obtained by the sensor cell 146.

The sensor element 12 further includes a reference gas adjustment pump cell 150. The reference gas adjustment pump cell 150 is an electrochemical pump cell formed of the third substrate layer 64, the solid electrolyte layers 66 and 70, the spacer layer 68, the outer pump electrode 114, and the reference electrode 102. The reference gas adjustment pump cell 150 performs pumping as a voltage Vp3 applied by a variable power supply 152 connected between the outer pump electrode 114 and the reference electrode 102 causes a control current Ip3 to flow. The reference gas adjustment pump cell 150 can pump oxygen into the atmosphere introduction layer 100 located around the reference electrode 102, from the sensor element chamber 28 (see FIG. 1) located around the outer pump electrode 114. The voltage Vp3 of the variable power supply 152 is a DC voltage such that the control current Ip3 has a predetermined value, and is determined in advance. That is, the voltage Vp3 of the variable power supply 152 is determined in advance as a DC voltage such that the control current Ip3 becomes a DC current with a constant value.

In this gas sensor 10, the main pump cell 110 and the auxiliary pump cell 124 operate to supply, to the measurement pump cell 140, the measured gas whose oxygen partial pressure is kept at a constant low value. That is, the measured gas whose oxygen partial pressure is kept at a value that does not substantially affect the measurement of the concentration of nitrogen oxide is supplied to the measurement pump cell 140. Then, oxygen in an amount substantially proportional to the concentration of the nitrogen oxide in the measured gas is generated by reduction of the nitrogen oxide. The oxygen thus generated is pumped out by the measurement pump cell 140. Since the pump current Ip2 flows in accordance with the amount of oxygen pumped out by the measurement pump cell 140, the concentration of the nitrogen oxide in the measured gas can be detected based on the pump current Ip2.

The sensor element 12 further includes a heater unit 160 for heating the sensor element 12 and keeping the temperature thereof. The heater unit 160 serves to adjust the temperature of the sensor element 12. By heating the solid electrolyte provided in the sensor element 12, the oxygen ion conductivity of the solid electrolyte can be increased. The heater unit 160 includes a heater connector electrode 162, a heater 164, a through hole 166, a heater insulating layer 168, a pressure release hole 170, and a lead wire 172.

The heater connector electrode 162 is formed, for example, on the lower surface of the first substrate layer 60. By electrically connecting the heater connector electrode 162 to an external power supply, power can be supplied from the external power supply to the heater unit 160.

The heater 164 is sandwiched between the second substrate layer 62 and the third substrate layer 64 from above and below. The heater 164 is formed of, for example, an electric resistor. The heater 164 is connected to the heater connector electrode 162 via the lead wire 172 and the through hole 166. The heater 164 generates heat by being supplied with power from the outside via the heater connector electrode 162. The heater 164 can heat and keep the temperature of the solid electrolyte forming the sensor element 12.

In plan view, the region from the internal cavity 88 to the internal cavity 96 overlaps the region in which the heater 164 is formed. Therefore, a portion which needs to be activated, of the solid electrolyte provided in the sensor element 12 can be sufficiently activated by the heater 164.

The heater insulating layer 168 is formed so as to cover the upper surface, the lower surface, and the side surfaces of the heater 164. As the material of the heater insulating layer 168, for example, an insulator can be used. More specifically, for example, porous alumina or the like can be used as the material of the heater insulating layer 168. The heater insulating layer 168 is provided to ensure electrical insulation between the second substrate layer 62 and the heater 164 and electrical insulation between the third substrate layer 64 and the heater 164.

The pressure release hole 170 penetrates through the third substrate layer 64 and the atmosphere introduction layer 100 and communicates with the reference gas introduction space 98. The pressure release hole 170 is formed for the purpose of reducing an increase in internal pressure due to an increase in temperature of the heater insulating layer 168.

The variable power supplies 122, 132, 144, 152 and the like are actually connected to the respective electrodes via lead wires (not shown) formed in the sensor element 12, the connector 24 (see FIG. 1), and the lead wires 54 (see FIG. 1).

Another example of the gas sensor according to the present embodiment will be described with reference to FIG. 4. FIG. 4 is a cross-sectional view showing another example of the gas sensor according to the present embodiment.

In the example shown in FIG. 4, a diffusion control portion 90 is provided between the diffusion control portion 86 and the diffusion control portion 94. The internal cavity 88 is formed between the diffusion control portion 86 and the diffusion control portion 90. An internal cavity 92 is formed between the diffusion control portion 90 and the diffusion control portion 94. The internal cavity 92 communicates with the internal cavity 88 via the diffusion control portion 90. Further, the internal cavity 92 communicates with the internal cavity 96 via the diffusion control portion 94. The diffusion control portion 90 includes, for example, two slits. The longitudinal direction of the slits is, for example, a direction perpendicular to the drawing sheet of FIG. 4. In the example shown in FIG. 4, the pump electrode 112 is located in the internal cavity 88, and the oxygen detection electrode 126 is located in the internal cavity 92. That is, in the example shown in FIG. 4, the pump electrode 112 and the oxygen detection electrode 126 are disposed in the separate internal cavities 88 and 92, respectively. In the example shown in FIG. 4, the value of Y is greater than 100%. That is, in the example shown in FIG. 4, the ratio (L1/L2) of the distance L1 between the first position P1 and the second position P2 to the longitudinal dimension L2 of the pump electrode 112 is greater than 100%. The diffusion control portion 90 may be formed of a porous body.

In this manner, the pump electrode 112 may be disposed in the internal cavity 88, and the oxygen detection electrode 126 may be disposed in the internal cavity 92 located closer to the rear end side than the internal cavity 88 is.

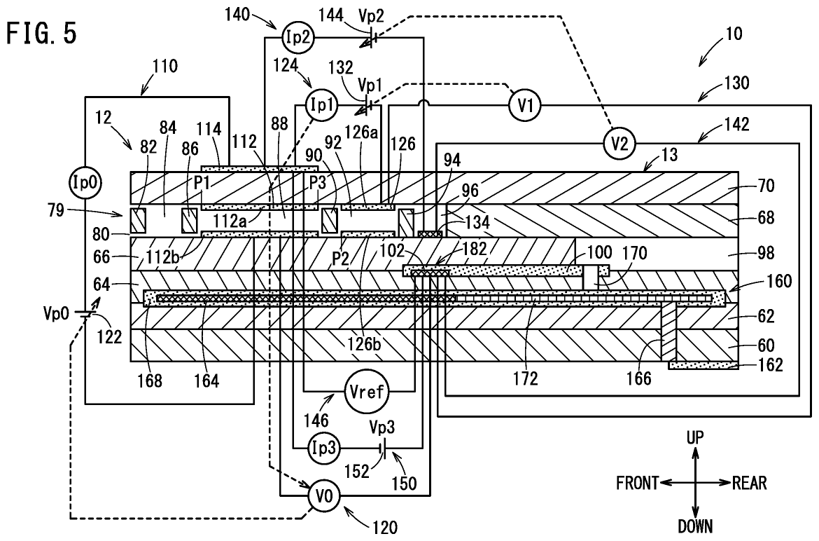

Still another example of the gas sensor according to the present embodiment will be described with reference to FIG. 5. FIG. 5 is a cross-sectional view showing still another example of the gas sensor according to the present embodiment.

In the example shown in FIG. 5, the pump electrode 112 is constituted by a plurality of electrodes respectively formed on the bottom surface of the internal cavity 88 and the top surface of the internal cavity 88. That is, the pump electrode 112 is constituted by a top pump electrode 112a and a bottom pump electrode 112b. The top pump electrode 112a and the bottom pump electrode 112b are electrically connected by patterns or the like (not shown). The top pump electrode 112a is formed on the top surface of the internal cavity 88. That is, the top pump electrode 112a is formed on the lower surface of the solid electrolyte layer 70. The bottom pump electrode 112b is formed on the bottom surface of the internal cavity 88. That is, the bottom pump electrode 112b is formed on the upper surface of the solid electrolyte layer 66.

In the example shown in FIG. 5, the oxygen detection electrode 126 is constituted by a top electrode portion 126a, a bottom electrode portion 126b, and side electrode portions (not shown). The top electrode portion 126a is formed on the top surface of the internal cavity 92. That is, the top electrode portion 126a is formed on the lower surface of the solid electrolyte layer 70. The bottom electrode portion 126b is formed on the bottom surface of the internal cavity 92. That is, the bottom electrode portion 126b is formed on the upper surface of the solid electrolyte layer 66. The side electrode portions are formed on side wall portions on both sides of the internal cavity 92. That is, the side electrode portions are formed on the side wall surfaces (inner surfaces) of the spacer layer 68. The top electrode portion 126a, the bottom electrode portion 126b, and the side electrode portions (not shown) are integrally formed. That is, the oxygen detection electrode 126 is formed in a tubular shape.

In the example shown in FIG. 5, as in the example shown in FIG. 4, the value of Y is greater than 100%. That is, in the example shown in FIG. 5, as in the example shown in FIG. 4, the ratio (L1/L2) of the distance L1 between the first position P1 and the second position P2 to the longitudinal dimension L2 of the pump electrode 112 is greater than 100%.

Thus, the pump electrode 112 may be formed on both the bottom surface of the internal cavity 88 and the top surface of the internal cavity 88. Further, the oxygen detection electrode 126 may be formed on both the bottom surface of the internal cavity 92 and the top surface of the internal cavity 92.

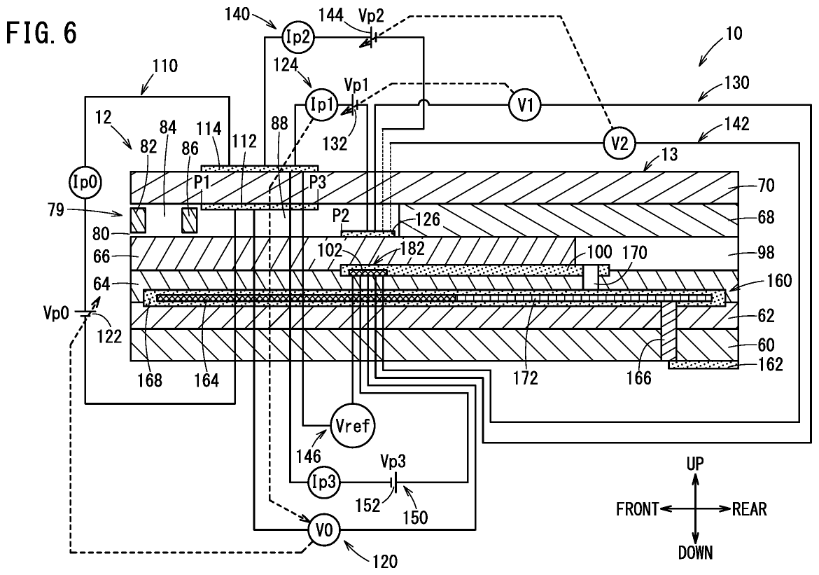

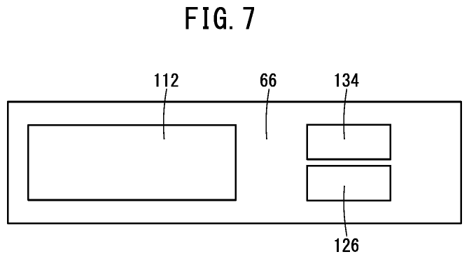

Yet another example of the gas sensor according to the present embodiment will be described with reference to FIG. 6. FIG. 6 is a cross-sectional view showing yet another example of the gas sensor according to the present embodiment. FIG. 7 is a plan view corresponding to a part of FIG. 6.

As shown in FIGS. 6 and 7, the pump electrode 112, the oxygen detection electrode 126, and the nitrogen oxide detection electrode 134 are disposed in the same internal cavity 88. The pump electrode 112 is formed on one of the bottom surface of the internal cavity 88 and the top surface of the internal cavity 88. The oxygen detection electrode 126 is formed on the other of the bottom surface of the internal cavity 88 and the top surface of the internal cavity 88. FIG. 6 shows an example in which the pump electrode 112 is formed on the top surface of the internal cavity 88 and the oxygen detection electrode 126 is formed on the bottom surface of the internal cavity 88. In the examples shown in FIGS. 6 and 7, the diffusion control portion 90 (see FIG. 4) and the diffusion control portion 94 (see FIG. 4) are not provided. As shown in FIG. 7, the nitrogen oxide detection electrode 134 is disposed along the flow direction of the measured gas in the measured gas flow path 79, and in parallel with the oxygen detection electrode 126. That is, the nitrogen oxide detection electrode 134 and the oxygen detection electrode 126 are disposed on both sides of the center line of the measured gas flow path 79 in the longitudinal direction.

In this way, the pump electrode 112, the oxygen detection electrode 126, and the nitrogen oxide detection electrode 134 may be disposed in the same internal cavity 88. Further, in this way, the nitrogen oxide detection electrode 134 and the oxygen detection electrode 126 may be disposed in parallel with each other.

### EXAMPLES

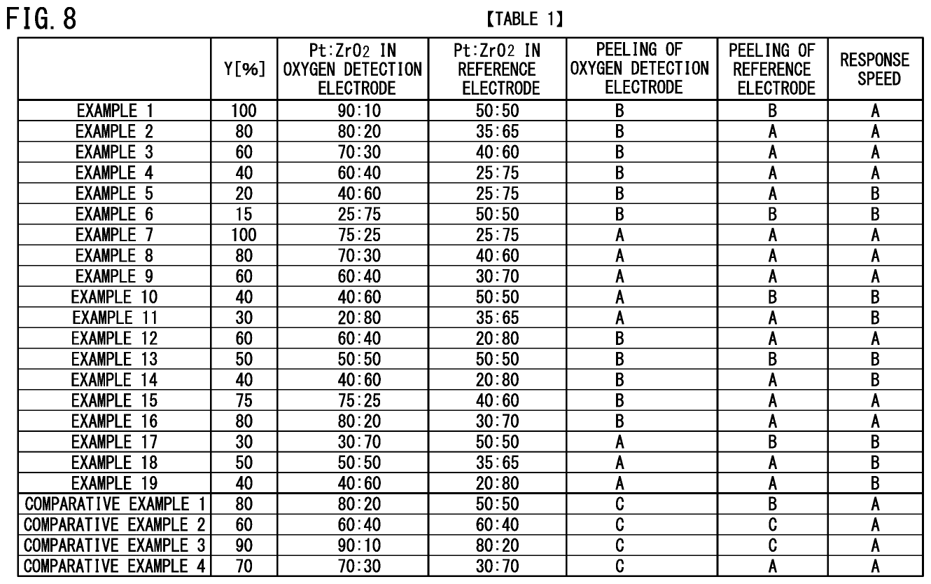

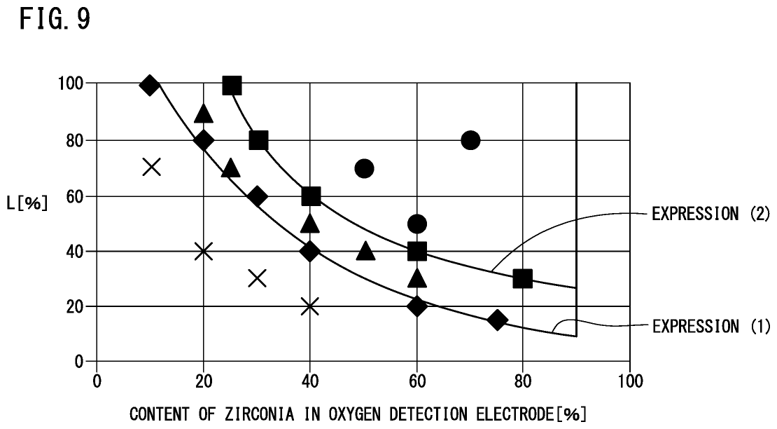

In Examples 1 to 19 and Comparative Examples 1 to 4, a peeling test for the oxygen detection electrode 126 and the reference electrode 102, and a response speed test were performed. The test results are shown in FIGS. 8 and 9. FIG. 8 is a diagram showing Table 1 illustrating the test results.

The peeling test for the oxygen detection electrode 126 and the reference electrode 102 was performed as follows. Specifically, the gas sensor 10 was placed in an air atmosphere at room temperature, and a test cycle including an ON state for 70 seconds and an OFF state for 50 seconds following the ON state was repeated 100,000 times. In the ON state, a predetermined voltage was applied to each part of the gas sensor 10. In the OFF state, no voltage was applied to each part of the gas sensor 10. In the ON state, power was supplied to the heater 164. In the ON state, signals were transmitted to and received from the gas sensor 10. In the OFF state, power supply to the heater 164 was stopped. In the OFF state, transmission and reception of signals to and from the gas sensor 10 were stopped. In the ON state, the main pump cell 110 was operated. In the ON state, oxygen was pumped in by applying a voltage across the outer pump electrode 114 and the reference electrode 102. A control current Ip3 flowing between the outer pump electrode 114 and the reference electrode 102 was set to 20 μA. After the peeling test was completed, the oxygen detection electrode 126 and the reference electrode 102 were observed. When the oxygen detection electrode 126 and the reference electrode 102 were observed, X-ray CT was used. Further, when the oxygen detection electrode 126 and the reference electrode 102 were observed, these electrodes were cut as necessary.

Evaluation criteria for peeling of the oxygen detection electrode 126 are as follows. Floating of the oxygen detection electrode 126 means that a gap is formed between the oxygen detection electrode 126 and the inner surface of the measured gas flow path 79.

A: Neither peeling nor floating occurs in the oxygen detection electrode 126.

B: Peeling does not occur in the oxygen detection electrode 126, but floating occurs in 50% or less of the oxygen detection electrode 126.

C: Peeling occurs in the oxygen detection electrode 126, or peeling does not occur in the oxygen detection electrode 126 but floating occurs in more than 50% of the oxygen detection electrode 126.

Evaluation criteria for peeling of the reference electrode 102 are as follows. Floating of the reference electrode 102 means that a gap is formed between the reference electrode 102 and the inner surface of the measured gas flow path 79.

A: Neither peeling nor floating occurs in the reference electrode 102. B: Peeling does not occur in the reference electrode 102, but floating occurs in 50% or less of the reference electrode 102.

C: Peeling occurs in the reference electrode 102, or peeling does not occur in the reference electrode 102 but floating occurs in more than 50% of the reference electrode 102.

The response speed test was performed as follows. First, the gas sensor 10 was attached to a test chamber. The response speed of the gas sensor 10 was measured by switching an excess air ratio X three times from 1.1 to 1.3 in a state in which feedback control of the pump voltage Vp0 based on the electromotive force V0 was not performed.

Evaluation criteria for the response speed are as follows.

A: The response speed is equal to or less than 500 ms.

B: The response speed is greater than 500 ms.

### Example 1

In Example 1, Y was set to 100%. As described above, Y is the ratio (L1/L2) of the distance L1 between the first position P1 and the second position P2 to the longitudinal dimension L2 of the pump electrode 112. As described above, the first position P1 is the position of the end portion of the pump electrode 112 on the front end side. As described above, the second position P2 is the position of the end portion of the oxygen detection electrode 126 on the front end side. The fact that Y is 100% means that the third position P3, which is the position of the end portion of the pump electrode 112 on the rear end side, and the second position P2, which is the position of the end portion of the oxygen detection electrode 126 on the front end side coincide with each other in plan view. The ratio (volume ratio) between the content of platinum and the content of zirconia in the oxygen detection electrode 126 was 90:10. The ratio between the content of platinum and the content of zirconia in the reference electrode 102 was 50:50. The evaluation result of the peeling test for the oxygen detection electrode 126 was B. The evaluation result of the peeling test for the reference electrode 102 was B. The evaluation result of the response speed test was A.

### Example 2

In Example 2, Y was set to 80%. The ratio between the content of platinum and the content of zirconia in the oxygen detection electrode 126 was 80:20. The ratio between the content of platinum and the content of zirconia in the reference electrode 102 was 35:65. The evaluation result of the peeling test for the oxygen detection electrode 126 was B. The evaluation result of the peeling test for the reference electrode 102 was A. The evaluation result of the response speed test was A.

### Example 3

In Example 3, Y was set to 60%. The ratio between the content of platinum and the content of zirconia in the oxygen detection electrode 126 was 70:30. The ratio between the content of platinum and the content of zirconia in the reference electrode 102 was 40:60. The evaluation result of the peeling test for the oxygen detection electrode 126 was B. The evaluation result of the peeling test for the reference electrode 102 was A. The evaluation result of the response speed test was A.

### Example 4

In Example 4, Y was set to 40%. The ratio between the content of platinum and the content of zirconia in the oxygen detection electrode 126 was 60:40. The ratio between the content of platinum and the content of zirconia in the reference electrode 102 was 25:75. The evaluation result of the peeling test for the oxygen detection electrode 126 was B. The evaluation result of the peeling test for the reference electrode 102 was A. The evaluation result of the response speed test was A.

### Example 5

In Example 5, Y was set to 20%. The ratio between the content of platinum and the content of zirconia in the oxygen detection electrode 126 was 40:60. The ratio between the content of platinum and the content of zirconia in the reference electrode 102 was 25:75. The evaluation result of the peeling test for the oxygen detection electrode 126 was B. The evaluation result of the peeling test for the reference electrode 102 was A. The evaluation result of the response speed test was B.

### Example 6

In Example 6, Y was set to 15%. The ratio between the content of platinum and the content of zirconia in the oxygen detection electrode 126 was 25:75. The ratio between the content of platinum and the content of zirconia in the reference electrode 102 was 50:50. The evaluation result of the peeling test for the oxygen detection electrode 126 was B. The evaluation result of the peeling test for the reference electrode 102 was B. The evaluation result of the response speed test was B.

### Example 7

In Example 7, Y was set to 100%. The ratio between the content of platinum and the content of zirconia in the oxygen detection electrode 126 was 75:25. The ratio between the content of platinum and the content of zirconia in the reference electrode 102 was 25:75. The evaluation result of the peeling test for the oxygen detection electrode 126 was A. The evaluation result of the peeling test for the reference electrode 102 was A. The evaluation result of the response speed test was A.

### Example 8

In Example 8, Y was set to 80%. The ratio between the content of platinum and the content of zirconia in the oxygen detection electrode 126 was 70:30. The ratio between the content of platinum and the content of zirconia in the reference electrode 102 was 40:60. The evaluation result of the peeling test for the oxygen detection electrode 126 was A. The evaluation result of the peeling test for the reference electrode 102 was A. The evaluation result of the response speed test was A.

### Example 9

In Example 9, Y was set to 60%. The ratio between the content of platinum and the content of zirconia in the oxygen detection electrode 126 was 60:40. The ratio between the content of platinum and the content of zirconia in the reference electrode 102 was 30:70. The evaluation result of the peeling test for the oxygen detection electrode 126 was A. The evaluation result of the peeling test for the reference electrode 102 was A. The evaluation result of the response speed test was A.

### Example 10

In Example 10, Y was set to 40%. The ratio between the content of platinum and the content of zirconia in the oxygen detection electrode 126 was 40:60. The ratio between the content of platinum and the content of zirconia in the reference electrode 102 was 50:50. The evaluation result of the peeling test for the oxygen detection electrode 126 was A. The evaluation result of the peeling test for the reference electrode 102 was B. The evaluation result of the response speed test was B.

### Example 11

In Example 11, Y was set to 30%. The ratio between the content of platinum and the content of zirconia in the oxygen detection electrode 126 was 20:80. The ratio between the content of platinum and the content of zirconia in the reference electrode 102 was 35:65. The evaluation result of the peeling test for the oxygen detection electrode 126 was A. The evaluation result of the peeling test for the reference electrode 102 was A. The evaluation result of the response speed test was B.

### Example 12

In Example 12, Y was set to 60%. The ratio between the content of platinum and the content of zirconia in the oxygen detection electrode 126 was 60:40. The ratio between the content of platinum and the content of zirconia in the reference electrode 102 was 20:80. The evaluation result of the peeling test for the oxygen detection electrode 126 was B. The evaluation result of the peeling test for the reference electrode 102 was A. The evaluation result of the response speed test was A.

### Example 13

In Example 13, Y was set to 50%. The ratio between the content of platinum and the content of zirconia in the oxygen detection electrode 126 was 50:50. The ratio between the content of platinum and the content of zirconia in the reference electrode 102 was 50:50. The evaluation result of the peeling test for the oxygen detection electrode 126 was B. The evaluation result of the peeling test for the reference electrode 102 was B. The evaluation result of the response speed test was B.

### Example 14

In Example 14, Y was set to 40%. The ratio between the content of platinum and the content of zirconia in the oxygen detection electrode 126 was 40:60. The ratio between the content of platinum and the content of zirconia in the reference electrode 102 was 20:80. The evaluation result of the peeling test for the oxygen detection electrode 126 was B. The evaluation result of the peeling test for the reference electrode 102 was A. The evaluation result of the response speed test was B.

### Example 15

In Example 15, Y was set to 75%. The ratio between the content of platinum and the content of zirconia in the oxygen detection electrode 126 was 75:25. The ratio between the content of platinum and the content of zirconia in the reference electrode 102 was 40:60. The evaluation result of the peeling test for the oxygen detection electrode 126 was B. The evaluation result of the peeling test for the reference electrode 102 was A. The evaluation result of the response speed test was A.

### Example 16

In Example 16, Y was set to 80%. The ratio between the content of platinum and the content of zirconia in the oxygen detection electrode 126 was 80:20. The ratio between the content of platinum and the content of zirconia in the reference electrode 102 was 30:70. The evaluation result of the peeling test for the oxygen detection electrode 126 was B. The evaluation result of the peeling test for the reference electrode 102 was A. The evaluation result of the response speed test was A.

### Example 17

In Example 17, Y was set to 30%. The ratio between the content of platinum and the content of zirconia in the oxygen detection electrode 126 was 30:70. The ratio between the content of platinum and the content of zirconia in the reference electrode 102 was 50:50. The evaluation result of the peeling test for the oxygen detection electrode 126 was A. The evaluation result of the peeling test for the reference electrode 102 was B. The evaluation result of the response speed test was B.

### Example 18

In Example 18, Y was set to 50%. The ratio between the content of platinum and the content of zirconia in the oxygen detection electrode 126 was 50:50. The ratio between the content of platinum and the content of zirconia in the reference electrode 102 was 35:65. The evaluation result of the peeling test for the oxygen detection electrode 126 was A. The evaluation result of the peeling test for the reference electrode 102 was A. The evaluation result of the response speed test was B.

### Example 19

In Example 19, Y was set to 40%. The ratio between the content of platinum and the content of zirconia in the oxygen detection electrode 126 was 40:60. The ratio between the content of platinum and the content of zirconia in the reference electrode 102 was 20:80. The evaluation result of the peeling test for the oxygen detection electrode 126 was A. The evaluation result of the peeling test for the reference electrode 102 was A. The evaluation result of the response speed test was B.

### Comparative Example 1

In Comparative Example 1, Y was set to 80%. The ratio between the content of platinum and the content of zirconia in the oxygen detection electrode 126 was 80:20. The ratio between the content of platinum and the content of zirconia in the reference electrode 102 was 50:50. The evaluation result of the peeling test for the oxygen detection electrode 126 was C. The evaluation result of the peeling test for the reference electrode 102 was B. The evaluation result of the response speed test was A.

### Comparative Example 2

In Comparative Example 2, Y was set to 60%. The ratio between the content of platinum and the content of zirconia in the oxygen detection electrode 126 was 60:40. The ratio between the content of platinum and the content of zirconia in the reference electrode 102 was 60:40. The evaluation result of the peeling test for the oxygen detection electrode 126 was C. The evaluation result of the peeling test for the reference electrode 102 was C. The evaluation result of the response speed test was A.

### Comparative Example 3

In Comparative Example 3, Y was set to 90%. The ratio between the content of platinum and the content of zirconia in the oxygen detection electrode 126 was 90:10. The ratio between the content of platinum and the content of zirconia in the reference electrode 102 was 80:20. The evaluation result of the peeling test for the oxygen detection electrode 126 was C. The evaluation result of the peeling test for the reference electrode 102 was C. The evaluation result of the response speed test was A.

### Comparative Example 4

In Comparative Example 4, Y was set to 70%. The ratio between the content of platinum and the content of zirconia in the oxygen detection electrode 126 was 70:30. The ratio between the content of platinum and the content of zirconia in the reference electrode 102 was 30:70. The evaluation result of the peeling test for the oxygen detection electrode 126 was C. The evaluation result of the peeling test for the reference electrode 102 was A. The evaluation result of the response speed test was A.

FIG. 9 is a graph showing test results. x marks are located in a non-acceptable region. Black rhombi correspond to Examples 1 to 6, and are located at the boundary between the non-acceptable region and a preferable region. Black triangles correspond to Examples 12 to 16, and are located within the preferable region. Black squares correspond to Examples 7 to 11, and are located at the boundary between the preferable region and a more preferable region. Black circles correspond to Examples 17 to 19, and are located within the more preferable region.

When an approximate curve of the black rhombi was obtained, the above-described expression (1) was obtained. The coefficient of determination (R²) in expression (1) is 0.9914. When an approximate curve of the black squares was obtained, the above-described expression (2) was obtained. The coefficient of determination (R²) in expression (2) is 0.9992.

It can be understood from the above-described test results that peeling of the oxygen detection electrode 126 can be suppressed if the positional relationship between the pump electrode 112 and the oxygen detection electrode 126, and the content of zirconia in the oxygen detection electrode 126 are set so as to satisfy the condition represented by expression (1). Further, it can be understood from the above-described test results that peeling of the oxygen detection electrode 126 can be further suppressed by satisfying the condition represented by expression (2).

In addition, it can be understood from the above-described test results that peeling of the reference electrode 102 can be suppressed by setting the content of zirconia in the reference electrode 102 to be equal to or higher than the content of platinum in the reference electrode 102.

It can also be understood from the above-described test results that a favorable response speed can be obtained by setting the content of platinum in the oxygen detection electrode 126 to be relatively high.

As described above, in the present embodiment, the positional relationship between the pump electrode 112 and the oxygen detection electrode 126, and the content X [%] of zirconia in the oxygen detection electrode 126 satisfy the relationship of Y≥141.96e^{-0.031X}. Y [%] is the ratio of the distance L1 between the first position P1 and the second position P2 to the longitudinal dimension L2 of the pump electrode 112. According to the present embodiment, since the positional relationship between the pump electrode 112 and the oxygen detection electrode 126, and the content of zirconia in the oxygen detection electrode 126 are set so as to satisfy such a relationship, peeling of the oxygen detection electrode 126 can be suppressed. Further, in the present embodiment, since the content of zirconia in the reference electrode 102 is equal to or higher than the content of platinum in the reference electrode 102, peeling of the reference electrode 102 can be suppressed. Therefore, according to the present embodiment, it is possible to provide the gas sensor 10 capable of suppressing the peeling of the oxygen detection electrode 126 and the reference electrode 102.

### [Modification]

Although the preferred embodiment of the present invention has been described above, the present invention is not limited to the above-described embodiment, and various modifications can be made thereto without departing from the scope of the present invention.

For example, in the above-described embodiment, the case where the auxiliary pump cell 124 is provided in the sensor element 12 and the oxygen detection electrode 126 can function also as an auxiliary pump electrode has been described as an example, but the present invention is not limited thereto. For example, the auxiliary pump cell 124 may not be provided in the sensor element 12, and the oxygen detection electrode 126 may not function as an auxiliary pump electrode. That is, the oxygen detection electrode 126 may not be an electrode constituting a part of the auxiliary pump cell 124.

The embodiments described above can be summarized as follows.

A gas sensor (10) comprises: a measured gas flow path (79) through which a measured gas introduced through a gas inlet (80) flows, the gas inlet being located on a front end side which is one side; a pump electrode (112) disposed in the measured gas flow path along a flow direction of the measured gas in the measured gas flow path; an oxygen detection electrode (126) disposed in the measured gas flow path and containing platinum and zirconia; and a reference electrode (102) disposed in a reference gas chamber (182) in which a reference gas exists, the reference electrode containing platinum and zirconia, wherein: when a position of a front end of the pump electrode is defined as a first position (P1), and a position of a front end of the oxygen detection electrode is defined as a second position (P2), the second position is located closer to a rear end side than the first position is, the rear end side being an opposite side to the front end side; when a content of zirconia in the oxygen detection electrode is defined as X [%], and a ratio (L1/L2) of a distance (L1) between the first position and the second position to a longitudinal dimension (L2) of the pump electrode is defined as Y [%], Y≥141.96e^{-0.031X} is satisfied; and a content of zirconia in the reference electrode is equal to or higher than a content of platinum in the reference electrode. According to such a configuration, since the positional relationship between the pump electrode and the oxygen detection electrode, and the content of zirconia in the oxygen detection electrode are set so as to satisfy the relationship of Y≥141.96e^{-0.031X}, peeling of the oxygen detection electrode can be suppressed. Further, according to such a configuration, since the content of zirconia in the reference electrode is equal to or higher than the content of platinum in the reference electrode, peeling of the reference electrode can be suppressed. Therefore, according to such a configuration, it is possible to provide a gas sensor capable of suppressing peeling of the oxygen detection electrode and the reference electrode.

Y≥2645.5X^{-1.024} may be satisfied. According to such a configuration, peeling of the oxygen detection electrode can be more reliably suppressed.

A content of platinum in the oxygen detection electrode may be higher than the content of zirconia in the oxygen detection electrode. According to such a configuration, since the content of platinum is relatively high, a gas sensor having a good response speed can be obtained.

The measured gas flow path may include an internal cavity (88) defined by diffusion control portions (86, 94), and the pump electrode and the oxygen detection electrode may be disposed in the same internal cavity provided in the measured gas flow path.

The pump electrode may be disposed on one of a top surface and a bottom surface of the internal cavity, and the oxygen detection electrode may be disposed on another of the top surface and the bottom surface of the internal cavity.

The measured gas flow path may include a plurality of internal cavities (88, 92) defined by diffusion control portions (86, 90, 94), the pump electrode may be disposed in a first internal cavity (88) among the plurality of internal cavities, and the oxygen detection electrode may be disposed in a second internal cavity (92) located closer to the rear end side than the first internal cavity is.

The gas sensor may further include a nitrogen oxide detection electrode (134) disposed in the measured gas flow path along the flow direction and in parallel with the oxygen detection electrode.

What is claimed is:

1. A gas sensor comprising:
a measured gas flow path through which a measured gas introduced through a gas inlet flows, the gas inlet being located on a front end side which is one side;
a pump electrode disposed in the measured gas flow path along a flow direction of the measured gas in the measured gas flow path;
an oxygen detection electrode disposed in the measured gas flow path and containing platinum and zirconia; and
a reference electrode disposed in a reference gas chamber in which a reference gas exists, the reference electrode containing platinum and zirconia, wherein:
when a position of a front end of the pump electrode is defined as a first position, and a position of a front end of the oxygen detection electrode is defined as a second position, the second position is located closer to a rear end side than the first position is, the rear end side being an opposite side to the front end side;
when a content of zirconia in the oxygen detection electrode is defined as X [%], and a ratio of a distance between the first position and the second position to a longitudinal dimension of the pump electrode is defined as Y [%],
Y≥141.96e^{-0.031X} is satisfied; and
a content of zirconia in the reference electrode is equal to or higher than a content of platinum in the reference electrode.

2. The gas sensor according to claim 1, wherein
Y≥2645.5X^{-1.024} is satisfied.

3. The gas sensor according to claim 1, wherein
a content of platinum in the oxygen detection electrode is higher than the content of zirconia in the oxygen detection electrode.

4. The gas sensor according to claim 1, wherein
the measured gas flow path includes an internal cavity defined by diffusion control portions, and
the pump electrode and the oxygen detection electrode are disposed in the same internal cavity provided in the measured gas flow path.

5. The gas sensor according to claim 4, wherein
the pump electrode is disposed on one of a top surface and a bottom surface of the internal cavity, and
the oxygen detection electrode is disposed on another of the top surface and the bottom surface of the internal cavity.

6. The gas sensor according to claim 1, wherein
the measured gas flow path includes a plurality of internal cavities defined by diffusion control portions,
the pump electrode is disposed in a first internal cavity among the plurality of internal cavities, and
the oxygen detection electrode is disposed in a second internal cavity located closer to the rear end side than the first internal cavity is.

7. The gas sensor according to claim 1, further comprising a nitrogen oxide detection electrode disposed in the measured gas flow path along the flow direction and in parallel with the oxygen detection electrode.
